(19) United States
(12) Patent Application Publication
(10) Pub. No.: US 2023/0210394 A1
(43) Pub. Date: Jul. 6, 2023

(54) ANALYSIS OF AN ACOUSTIC SIGNAL

(71) Applicants: Fraunhofer-Gesellschaft zur Förderung der angewandten Forschung e.V., München (DE); Boehringer Ingelheim Vetmedica GmbH, Ingelheim am Rhein (DE)

(72) Inventors: Michael PANTFÖRDER, Dortmund (DE); Pinar BISGIN, Dortmund (DE); Christoph Matthias SCHUMMER, Ingelheim am Rhein (DE); Andrew BEARDOW, Duluth, GA (US)

(21) Appl. No.: 18/159,241

(22) Filed: Jan. 25, 2023

Related U.S. Application Data
(63) Continuation of application No. PCT/EP2021/071159, filed on Jul. 28, 2021.

(30) Foreign Application Priority Data
Jul. 31, 2020 (EP) 20188977.1

(51) Int. Cl. A61B 5/0255; A61B 5/00; A61B 7/04

(52) U.S. Cl. CPC A61B 5/0255; A61B 5/7264; A61B 7/04; A61B 2503/40

(57) ABSTRACT

A method for analyzing an acoustic signal having a time period and having a plurality of repeated audio patterns, has the following steps: receiving an audio signal having the acoustic signal; determining the audio patterns repeated within the acoustic signal; determining a window length for a plurality of windows, wherein the window length divides the time period of the acoustic signal into the plurality of windows; and windowing the acoustic signal to obtain the plurality of windows.

### CROSS-REFERENCE TO RELATED APPLICATIONS

This application is a continuation of copending International Application No. PCT/EP2021/071159, filed Jul. 28, 2021, which is incorporated herein by reference in its entirety, and additionally claims priority from European Application No. 20188977.1, filed Jul. 31, 2020, which is also incorporated herein by reference in its entirety.

### TECHNICAL FIELD

Embodiments of the present invention refer to a method for analyzing an acoustic signal and to a corresponding apparatus. Further embodiments refer to a system for performing an analysis comprising a respective apparatus. Further embodiments refer to a computer program.

An acoustic signal enables the determination of unwanted effects, like damaging of machinery or a disease of an animal, such as a non-human mammal, in particular a dog.

### BACKGROUND OF THE INVENTION

The following publications form known technology: Hebden J H et al., “Identification of aortic stenosis and mitral regulation of heart sound analysis”, Computers in Cardiology 1997, 24: 109-112; Zhang W et al., “Heart sound classification based on scaled spectrogram and partial least squares regression”, Biomedical Signal Processing and Control, 2017, 32: 20-28; Ari S et al., “Detection of cardiac abnormality from PCG signal using LMS based least square SVM classifier”, Expert Systems with Applications, 2010, 37: 8019-8026; Jamous G et al., “Optimal time-window duration for computing time/frequency representations of normal phonocardiograms in dogs”, Med. & Biol. Eng. & Comput., 1992, 30: 503-508; and Ismail S et al., “Localization and classification of heart beats in phonocardiography signals—a comprehensive review”, EURASIP Journal of Advances in Signal, 2018 (1): 26.

Here, it has been found that improvements for the analysis of the acoustic signal may lead to significant improvements for the determination of the damage or disease, e.g. regarding accuracy and reliability. Therefore, it is an objective of the present invention to improve acoustic analysis.

### SUMMARY

According to an embodiment, a method for analyzing an acoustic signal having a time period and having a plurality of repeated audio patterns may have the steps of: receiving an audio signal having the acoustic signal, wherein the audio signal is a record of a heartbeat sequence of an animal, advantageously a non-human mammal, more advantageously a dog, and/or a record of a heart murmur sequence of an animal, advantageously a non-human mammal, more advantageously a dog; determining the audio patterns repeated within the acoustic signal; determining a window length for a plurality of windows, wherein the window length divides the time period of the acoustic signal into the plurality of windows; wherein determining the window length is performed for each window of the plurality of windows separately; and windowing the acoustic signal to obtain the plurality of windows; wherein the step of determining the audio patterns, determining a window length and the windowing are performed automatically.

According to another embodiment, an apparatus for analyzing an acoustic signal having a time period and having a plurality of repeated audio patterns may have: an interface for receiving the audio signal having the acoustic signal; the audio signal is a record of a heartbeat sequence of an animal, advantageously a non-human mammal, more advantageously a dog and/or a record of a heart murmur sequence of an animal, advantageously a non-human mammal, more advantageously a dog; and a processor which is configured to determine the audio pattern repeated within the acoustic signal and to determine a window length for a plurality of windows, wherein the window length divides the time period of the acoustic signal into the plurality of windows, wherein the processor determines the window length for each window of the plurality of windows separately; and to window the acoustic signal to obtain the plurality of windows; wherein the step of determining the audio patterns, determining a window length and the windowing are performed automatically.

Another embodiment may have a system for performing an analysis having the above inventive apparatus and a microphone or advantageously the above inventive apparatus and a stethoscope having a microphone or more advantageously the above inventive apparatus and a digital stethoscope having a microphone.

Still another embodiment may have a non-transitory digital storage medium having stored thereon a computer program for performing a method for analyzing an acoustic signal having a time period and having a plurality of repeated audio patterns having the steps of: receiving an audio signal having the acoustic signal, wherein the audio signal is a record of a heartbeat sequence of an animal, advantageously a non-human mammal, more advantageously a dog, and/or a record of a heart murmur sequence of an animal, advantageously a non-human mammal, more advantageously a dog; determining the audio patterns repeated within the acoustic signal; determining a window length for a plurality of windows, wherein the window length divides the time period of the acoustic signal into the plurality of windows; wherein determining the window length is performed for each window of the plurality of windows separately; and windowing the acoustic signal to obtain the plurality of windows; wherein the step of determining the audio patterns, determining a window length and the windowing are performed automatically, when said computer program is run by a computer.

Embodiments of the present invention provide a method for analyzing an acoustic signal having a time period and comprising a plurality of repeated audio patterns, e.g., a periodic sound of a train passing a railway sleeper or a heartbeat of an animal, such as a non-human mammal, in particular a dog. The method comprises the following steps:
Receiving an audio signal, like an audio record, comprising the acoustic signal;
Determining the audio patterns repeated within the acoustic signal;
Determining a window length for a plurality of windows, wherein the window lengths divide the time period of the acoustic signal into the plurality of windows; and
Windowing the acoustic signal to obtain the plurality of windows

According to an embodiment, the method further comprises the step of analyzing the respective (separated) windows of the plurality of windows.

Embodiments of the present application are based on the finding that an acoustic signal, like a series of heartbeats or a series of side sounds resulting from a rotary machine have a periodicity. By knowing/determining this periodicity, the acoustic signal can be subdivided into a plurality of windows, such that each window comprises at least one of the repeated audio pattern. This enables to analyze each of the repeated audio patterns independent from the other, e.g., by comparing this audio pattern with a known audio pattern. Alternatively, the repeated audio pattern can be analyzed with respect to the other audio pattern subsequent to the respective audio pattern.

It should be noted that the repeated audio patterns may be equal to each other, substantially equal to each other, similar to each other, comprise one or more peaks of a comparable shape (shape of the respective amplitude plotted over the time) and/or comprise one or more peaks of comparable shape (shape of the altitude plotted over the time) and comparable amplitude values at respective points of time within the window length, etc. According to embodiments, the window length is equal. For example, the window lengths may be determined based on a frequency of the repetition of the repeated pattern. According to another variant, the borderline between two patterns is determined so as to determine the window length for the respective window. This means that each window length for each window is determined separately.

According to a further embodiments, the step of analyzing the respective windows comprises the step of performing a feature extraction to obtain one or more extracted features describing the respective pattern (of the window). According to embodiments, the features to be extracted are out of the group comprising a named feature, time domain feature and/or frequency domain feature.

Examples are:
a maximum, a mean, median, standard deviation, variance, skewness, kurtosis, mean absolute deviation, quantile 25th, quantile 75th, entropy, zero crossing rate, crest factor, duration of a first peak and/or second peak within the pattern, duration between the first peak and the second peak within the pattern, duration between the second peak of a first pattern and the first peak of a subsequent pattern, mel frequency cepstral coefficients, pitch chroma, spectral flatness, spectral kurtosis, spectral skewness, spectral slope, spectral entropy, dominant frequency, bandwidth, spectral centroid, spectral flux, spectral roll off, class information, severity information, position information, race information, weight information, additional information and/or other parameters or a combination thereof.

Additionally, and/or alternatively, the feature extraction may comprise a step of reducing the value range for the one or more extracted features so that the value range for the one or more extracted features is defined between a minimum value (e.g., 0) and a maximum value (e.g., 1).

It should be noted that according to embodiments, the audio pattern is defined by one or more peaks. Each repeated audio pattern may alternatively or additionally be defined by one or more peaks in combination with a basis wherein the one or more peaks have an amplitude value, which is at least five times larger than the basis level. Additionally/alternatively, each repeated audio pattern may be defined by a systole and/or diastole, e.g., when the acoustic signal is the heartbeat sequence of an animal, such as a non-human mammal, in particular a dog.

According to embodiments, the method comprises the step of normalizing the audio signal.

According to embodiments, the steps of determining the audio patterns, determining a window length and the windowing are performed automatically or performed by use of artificial intelligence. The steps may, for example, be performed by use of a decision tree algorithm, a random forest algorithm, a naive bayes algorithm, adaboost algorithm, and/or a support vector machine algorithm.

As indicated above, a possible application is the diagnosis of a disease for an animal, such as a non-human mammal, in particular a dog. Therefore according to embodiments, the acoustic signal/audio signal is a record of a heartbeat sequence of a dog or another animal or another non-human mammal, and/or a record of a heart murmur sequence of a dog or another animal or another non-human mammal.

Another embodiment provides an apparatus for analyzing an acoustic signal having a time period and comprising a plurality of repeated audio patterns. The apparatus comprises an interface for receiving the audio signal comprising the acoustic signal and a processor. The processor is configured to determine the audio pattern repeated within the acoustic signal and to determine a window length for a plurality of windows, wherein the window lengths divides a time period of the acoustic signal into the plurality of windows. Furthermore, the processor is configured to window the acoustic signal in order to obtain the plurality of windows. Another embodiment provides a system for performing an analysis comprising an apparatus and a microphone.

According to an embodiment, the system comprises the apparatus and a stethoscope comprising a microphone. According to another more advantageous variant, the system comprises the apparatus and a digital stethoscope comprising a microphone.

According to further embodiments, the above-described method may be computer implemented, therefore an embodiment refers to a computer program.

All embodiments may be used to medically examine an animal, especially a non-human mammal, like a dog or cat, in particular a dog.

### BRIEF DESCRIPTION OF THE DRAWINGS

Below, embodiments of the present invention will subsequently be discussed referring to the enclosed figures, in which:

FIG. 1a shows a schematic flow chart illustrating a method for analyzing an acoustic signal according to a basic embodiment;

FIG. 1b schematically shows the input and output signal of the steps discussed in the context of FIG. 1a according to further embodiments;

FIGS. 2a and 2b show an example of an acoustic signal to be processed according to an embodiment;

FIG. 3 illustrates schematically a certain processing step of an acoustic signal to illustrate embodiments;

FIGS. 4a and 4b schematically illustrate the issues occurring during processing an acoustic signal to discuss embodiments;

FIG. 5 shows a schematic block diagram of an apparatus for analyzing an acoustic signal; and

FIG. 6a-c show schematic patterns indicating different diseases.

### DETAILED DESCRIPTION OF THE INVENTION

Below, embodiments of the present invention will subsequently be discussed referring to the enclosed figures, wherein identical reference numerals are provided to objects having identical or similar functions, so that the description thereof is interchangeable and mutually applicable.

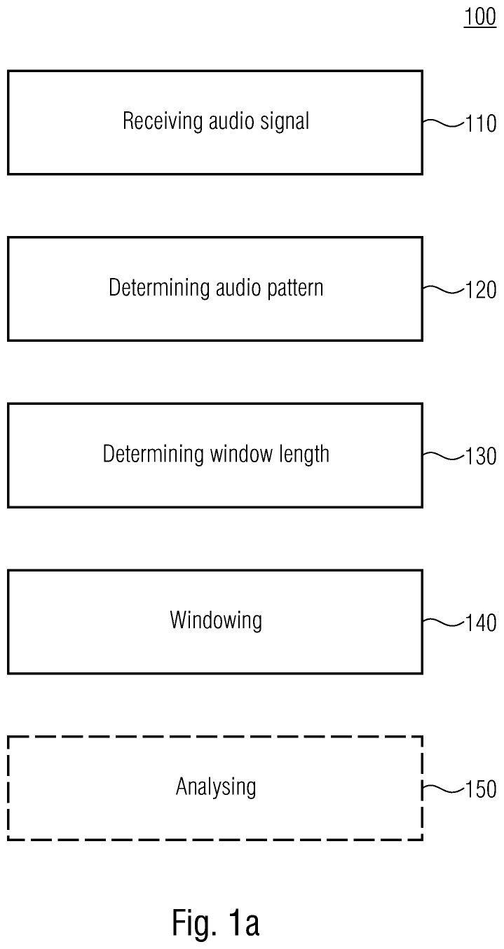

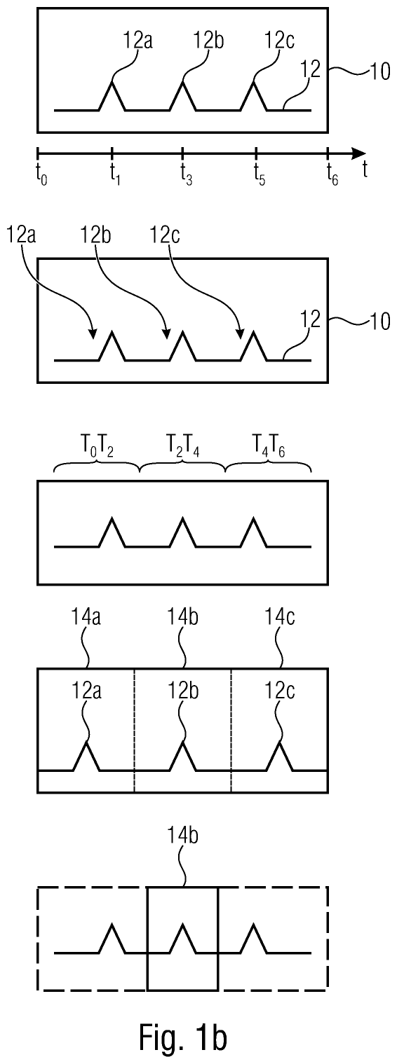

FIGS. 1a and 1b show the method 100. The method 100 comprises four basic steps and an optional step subsequent to the four basic steps.

The four basic steps are marked by the reference numerals 110, 120, 130, 140, wherein the optional step is marked by the reference numeral 150. The shown order is the advantageous order, but not the required.

In the first step 110 an audio signal 10 (cf. FIG. 1b) is received. The audio signal 10 comprises an acoustic signal 12 having a time period T0 to T6. The acoustic signal 12 comprises a plurality of repeated audio patterns that are marked by 12a, 12b and 12c at the points of time T1, T3 and T5.

Within the next step 120 the audio patterns 12a, 12b and 12c are identified/determined. For example, the determination may be based on an algorithm finding repetitions within (audio) signal. This algorithm may be based on artificial intelligence/self-learning algorithms.

Within the next step 130, a window length is determined. Window lengths are determined, such that it is as long as the single pattern 12a/12b/12c. For example, the entire time period T0 to T6 may be divided by the number of determined patterns 12a, 12b and 12c. By doing so, a window lengths of equaling window lengths for each pattern is determined. For example, the window lengths T0 to T2, T2 to T4, and T4 to T6 is determined. Based on this window length, the time period T0 to T6 is subdivided (cf. step 140). The result of this windowing step 140 is a plurality of windows marked by the reference numerals 14a, 14b and 14c. Here, the window 14a comprises the pattern 12a, the window 14b, the pattern 12b and the window 14c the pattern 12c.

After that, the optional step of analyzing 150 may follow. Here, the windows 14a, 14b and 14c are analyzed. For example, the window 14b is extracted and analyzed independent from the other windows, e.g., by performing feature extraction. This feature extraction may also be performed for the windows 14a and 14c as well. Additionally or alternatively, the window 14b may be compared to the other windows, e.g., the window 14a and 14b, in order to determine the regularity of the patterns.

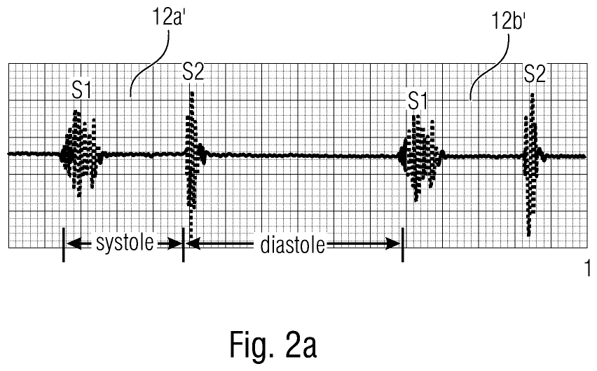

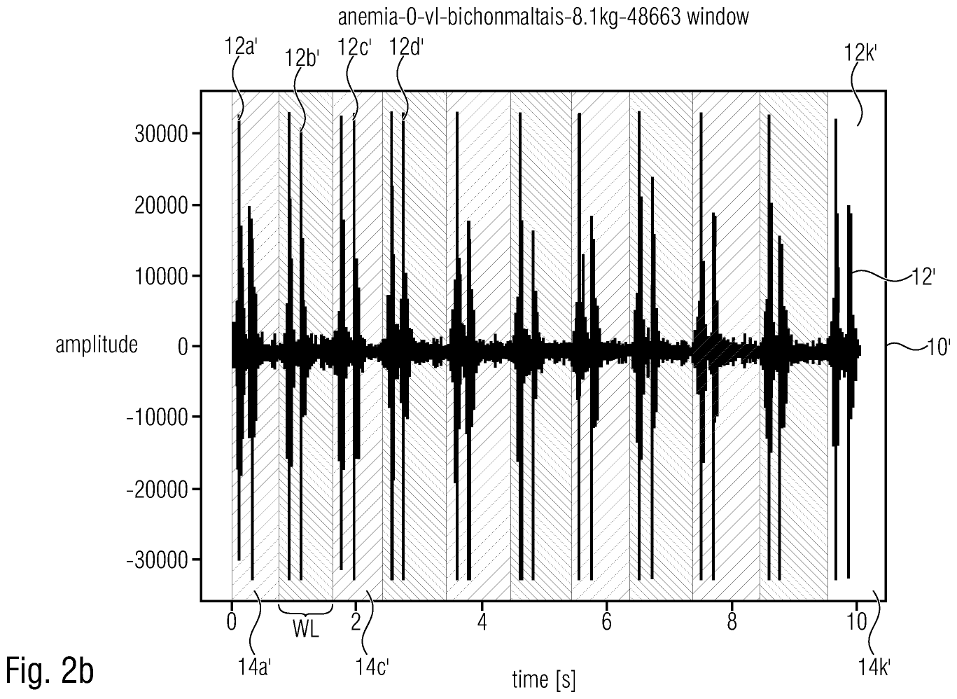

With respect to FIG. 2a, an exemplarily analysis for a heartbeat sequence will be discussed. FIG. 2a shows an audio signal 10′ comprising an acoustic signal 12′. This acoustic signal represents, for example, a sequence of heartbeats, e.g., of a dog. The duration of the record 10′ may be approximately 10 seconds, wherein these 10 seconds may comprise 11 heartbeat patterns marked with reference numeral 12a′, 12b′, etc., to 12k′. Alternatively, the duration may be at least 3 seconds, at least 5 seconds, at least 15 second, at least 30 seconds or at least 1 minute, in general 5 to 180 seconds or 1 to 300 seconds or at least 1 or at least 10 seconds. Each pattern 12a′, 12b′ may comprise two signals S1 and S2 as illustrated by FIG. 2b.

FIG. 2b shows an enlarged view, e.g., of the two patterns 12a′ and 12b′. The signal S1 may be the peak, indicative for the beginning of the systole, while S2 is a peak indicative for the beginning of the diastole. Each peak S1 and S2 is formed by an amplitude comparably high when compared to the basis signal. As can be seen, the fundamental structure of the pattern 12a′ is comparable to the structure of the pattern 12b′. This means that the amplitude of the peak S1 has approximately the same height, when the time interval between S1 and S2 is also comparable for the two patterns 12a′ and 12b′. As can be seen with respect to FIG. 2b, each pattern 12a′ and 12b′ may have two or more peak S1 and S2. Additionally, the pattern may have a base signal/zero signal between the two peaks S1 and S2. The combination of the two peaks S1 and S2 and the base signal between the two peaks and/or the base signal after the last peak S2 may define the pattern. At this stage, it should be mentioned that a pattern 12a′, 12b′, etc. may also be defined by just one peak and one base signal, or just two peaks without base signal in between, or by another combination.

In order to separate the patterns 12a′, 12b′, etc., a windowing is performed. From this, the window lengths are determined. The window lengths may be determined based on the duration of the acoustic signal 12′, here 10 seconds and the number of patterns 12a′, etc., here 11 patterns. The calculation may be performed by a simple division. In this example, the result would be that the window lengths for each window amounts to approximately 0.9 seconds. Of course, the window lengths may, according to further embodiments, be determined differently, e.g., by determining the duration of each pattern, i.e., the interval between S1 and the subsequent S1, and averaging these durations. According to further embodiments, the window lengths may vary over time, e.g., when the periodicity of the pattern varies. This can happen, e.g., when the heartbeat rate decreases in the current situation. In this example, the window lengths WL for all patterns 12a′ to 12k′ is equal. Therefore, 11 windows 14a′ to 14k′ are used to subdivide the audio signal 12′. Therefore, each window 14a′ to 14k′ comprises a respective pattern 12a′ to 12k′. This enables that within each window 14a′ to 14k′ a feature extraction can be performed, i.e., not for the entire record 10′ of the acoustic signal 12′, but for each pattern 12a′ to 12k′, or each heartbeat, respectively.

According to embodiments, the window length may be adapted, e. g. from a first window to a second window (subsequent window of the plurality of windows). It is resolved that each window length or the window length of at least two windows is different/varied. According to embodiments, an adaptation may be performed based on the determination of a heartbeat sound, like a systole (S1) or another characteristic feature within the pattern or a current heartbeat rate of the animal or non-human mammal, such as a dog or cat. Therefore the method may optionally comprise a step of determining a characteristic feature of the (heartbeat) pattern or the heartbeat rate so as to adapt the window length. As a consequence, the window length is dependent on the heartbeat rate. A result may be that the lengths of the heartbeat phase/pattern can be determined.

According to embodiments, this may have the purpose that comparable windows within which the analysis may be performed are obtained so that the respective position of the systole (S1, S2) or diastole within the respective window is achieved so that the analysis can be improved. The position of the murmur within the window/pattern/heartbeat phase is a relevant factor.

According to embodiments, the dynamical windowing may be performed using a wavelet transformation. For example, the peaks S1 and S2 within the audio signal are determined accurately so that each window can be set at a certain position with respect to such a peak S1 or S2, for example, at the beginning of each peak (increasing slope). Thus, according to embodiments, the beginning of each window is determined based on such a peak or a characteristic element of the pattern. According to further embodiments, the respective end of each window is determined analogously, e.g., at the beginning of the next comparable characteristic, e.g., the next comparable characteristic, e.g., the system S1. This means that according to embodiments, the windowing is performed by determining a respective characteristic within each pattern, wherein the characteristic of the first pattern is used as beginning of a respective window, while the end of said window is determined based on the respective characteristic of the subsequent window.

According to embodiments, the position of the murmur within the window enables to gain additional information on the disease. Therefore, the method further comprises the step of determining the position (time position) within the respective window of the murmur. For example, differentiation may be made whether the murmur is determined between the first systole S1 and the second systole S2, closer to the first systole S1 then to the second systole S2, closer to the second systole S2 then to the first systole S1, or behind the second systole S2. Examples for such diagnoses are: a systolic murmur deriving from the left heart chamber mitral valve (mitral valve regurgitation/leakage), or a systolic murmur deriving from a leaking tricuspid valve (right heart chamber), or a systolic murmur resulting from an aortic or pulmonary artery stenosis, or a permanent (both systolic and diastolic) murmur deriving from congenital diseases, such as a persistent ductus arteriosus, chamber or atrial wall defects. Also in diastole, murmurs can be auscultated, such as defects resulting from either valvular stenosis and/or valvular insufficiencies. These different diagnoses can be differentiated automatically due to their characteristic sound pattern.

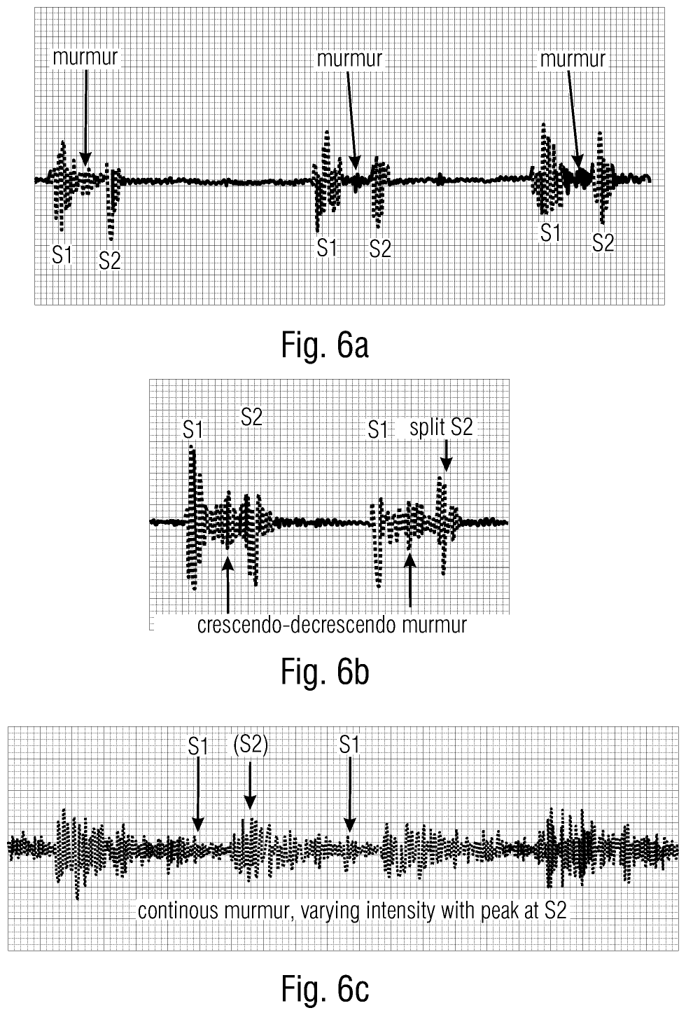

Examples of patterns indicating different diseases are shown by FIG. 6a-6c. FIG. 6a shows a typical pattern for mitral regurgitation (ME). Here, the murmur is between S1 and S2.

FIG. 6b shows a pulmonic stenosis murmur (PS). In this case the noise has a crescendo-decrescendo-character and extend across 50-85% of the systole.

FIG. 6c shows a persistent ductus arteriosus with left to right shunting of blood (PDA): During the systole a reduced, decreased or even reversed shunting can occur.

Another murmur is the so called dilated cardiomyopathy (DCM): Some dogs having DCM do not produce a noise, but an extension of the atrial valve annulus can cause a mitral and tricuspid regurgitation having a systolic noise (maximum intensity over the apex of the heart).

These are typical murmurs which can be determined using the algorithm.

According to further embodiments, different machine learning approaches may be used to categorize the patterns. Examples are random forest, support vector machines, neural networks, decision trees, random forest and AdaBoosts. For example, a detection of a mitral valve disease is advantageously done by use of a random forest or AdaBoost algorithm, while for other diseases, the used algorithm can vary.

According to embodiments, this extra information is determined automatically. Therefore, the method comprises the step of determining a diagnosis of the respective murmur/respective disease based on the position of the murmur within the window or, in general, based on the structure of the acoustic pattern. According to embodiments, the pattern is determined within a sequence of repeated patterns or extracted/separated from the sequence, wherein the sequence comprises a plurality of patterns which are equal or comparable to each other.

According to further embodiments, a feature extraction can be performed for each window 14a′ to 14k′ as will be discussed below. For example, an amplitude value can be extracted as feature. After that, the feature can be processed, e.g., by calculating the average value/median value.

In the short explanation:
Determining the window borderlines for the respective record
Determining the one or more features per window
Processing the one or more features, e.g., average determination, determination of the median
Proceed with the next record

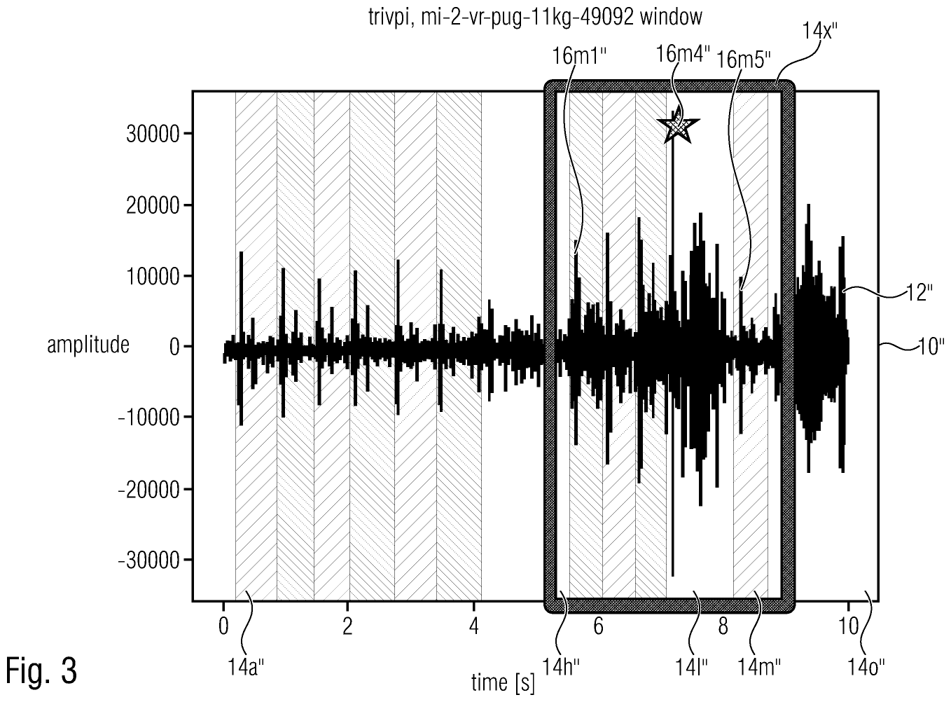

Below, with respect to FIG. 3, a feature extraction for the feature maximum will be discussed.

FIG. 3 shows an audio record 10″ comprising an acoustic signal 12″ which is subdivided into a plurality of windows 14a″ to 14o″ for simplification reasons, just the windows 14h″, 14m″ marked by the frame 14x″ will be taken into account. Here, the maximum amplitudes of the peaks are determined. These maximum amplitudes are marked by the reference numerals 16m1″ to 16m5″. The maximum 16m4″ amounts to approximately 100 and belongs to the window 141″. Since this maximum 14m″ is, when compared to the maximum 14m1″, 14m2″, 14m3″ and 14m5″ significantly higher and since the entire signal within the window 141″ seems to be disturbed, these values are not taken into account. The maximum feature of the windows 14h″, 14i″, 14j″ and 14m″ is approximately (15,000, 17,000, 19,000, 10,000). The mean feat max is 15,215, while the median: feat max=16.000. Instead of reducing all four features 16m1″, 14m2″, 14m3″ and 14m5″, all four features can be further used.

As illustrated with respect to FIG. 3, some pattern within the window are not usable.

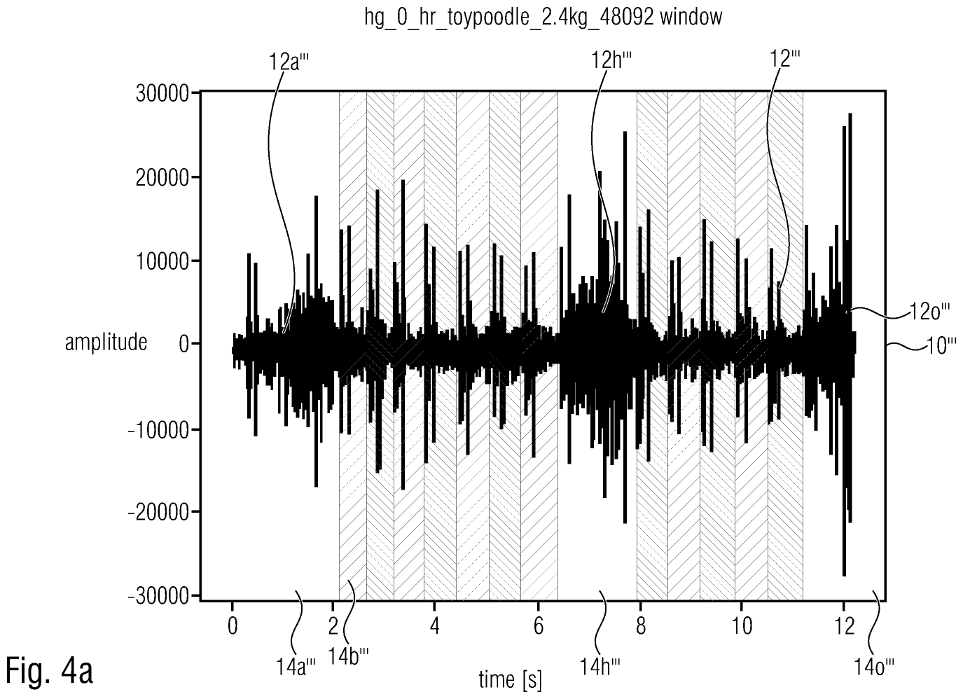

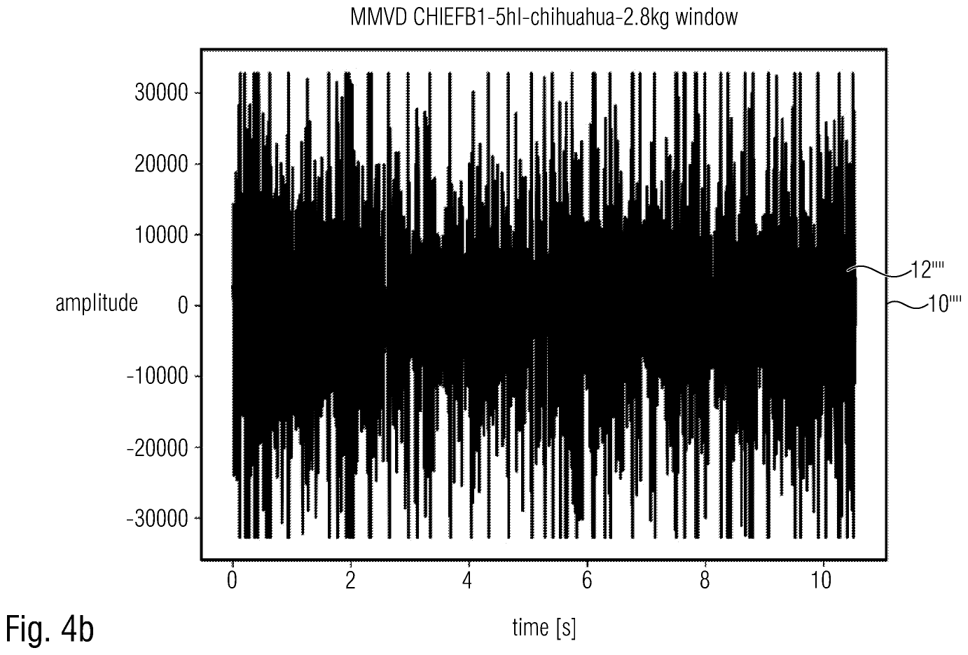

This will be illustrated with respect to FIG. 4a. FIG. 4a shows an audio signal 10′″ comprising an acoustic signal 12′″ which should be subdivided into a plurality of windows. Here, some windows 14a′″ to 14o′″ are illustrated. Especially for the windows 14a′″, 14h″ and 14o′″ the respective pattern 12a′″, 12h′″ and 12o′″ is disturbed. Therefore, these windows 14a′″, 14h′″ and 14o′″ are not used/neglected. In contrast to the disturbed window 141″ of FIG. 3, which has a too high maximum value, the reason for neglecting the windows 14a′″, 14h′″ and 14o′″ is that the borderline between the previous and subsequent windows cannot be determined. Since such disturbed signals of the windows 14a′″, 14h′″ and 14o′″ or 141″ would falsify the result of the feature extraction, the respective windows 14a′″, 14h′″, 14o′″ and 141″ are neglected. With respect to the embodiment of FIG. 4a, this means that all windows 14a′″, 14h′″ and 14o′″ are neglected, for which no clear borderline can be determined.

FIG. 4b shows another disruption. Here, the entire acoustic signal 12″″ comprises amplitude values that are out of the range and have no clear signals, such that a windowing cannot be performed. Therefore, in some cases, the entire record 10″″ comprising the completely disturbed signal 12″″ may be neglected without windowing.

Below, a possible analysis step of the respective windows 14a, 14b and 14c (cf. FIG. 1a and 1b) or of other windows belonging to other embodiments will be discussed. The windowing enables that each window 14a, 14b and 14c and each pattern 12a, 12b and 12c can be analyzed separately.

According to embodiments, three different types of features can be extracted, namely name features, time domain features and frequency domain features. The time domain features and the pfrequency domain features mainly refer to the acoustic signal 12a, 12b and 12c, while the main feature refers to a side information. Possible time domain features which can be analyzed for each window 14a, 14b and 14c are:

The mean of the pattern, the median of the pattern, the standard deviation within the pattern, the variance within the pattern or with respect to another pattern, the skewness of the pattern, the kurtosis of the pattern the mean absolute deviation of the patent, the quantile 25th of the pattern, the quantile 75th of the pattern, the entropy of the pattern, the 0 crossing rate within the pattern, the quest factor, the duration of the first peak as 1, the duration of the other peak S2, the duration from the end of S1 to the start of S1, the duration of end of S2 to the start of the next S1. Especially, the duration features are more meaningful when the signal 12 is windowed into the window 14a to 14c.

Time domain features can be the mel frequency cepstral coefficients, the pitch chroma, the spectral flatness, the spectral kurtosis, the spectral skewness, the spectral slope, the spectral entropy, the dominant frequency, the bandwidth, the spectral centroid, the spectral flux, and/or the spectral roll off.

As discussed above, an example for an audio signal may be the heartbeat of an animal or a non-human mammal, like a dog. Due to this, the possibility exists that additional information can be taken into account, namely so-called name features. Name features may be the class, the severity (severity for the disease in steps 0-6), the position (measurement position at the animal, for example, front left, front right, back left, back right), the race, the weight (weight classes may be used, e.g., 0-10 kg, 10-20 kg, ≥20 kg). Additionally, it is possible that a node can be taken, e.g., post operation or prior operation).

It should be noted that the lists for the different feature types and the feature type is not limited to the mentioned ones.

According to embodiments, the above analysis step is mainly or completely performed automatically. Especially the windowing may be performed automatically (of the windowing). During the learning phase, the windowing and an exemplary analysis may be performed. For the learning phase, the parameters may be set for the windowing/auto-windowing, for the feature list (especially for usage of the windowing, here the mean/median or all may be used for the analysis (and the test size) the percentage of the test data set (e.g., 0.3). here, the test data set is split and randomized, wherein for example 30% is used for the learning. For the learning, different models can be used, e.g., a decision tree model, a random forest model, a naive bayes base model, an adaboost model, and/or a support vector machine model. It has been found that the random forest model enables the best results.

As discussed above, the discussed approach may be used for a diagnosis of an animal or a non-human mammal, e.g. a dog. Below, the background will be discussed. Mitral valve endocardiosis is the most common heart disease in dogs. The prevalence increases with age, approximately 10% of all 5 to 8-year-old dogs, approximately 25% of all 9 to 12-year-old dogs and 35% of all dogs over 13 years of age are affected. Mainly older dogs of small breeds are affected, such as: toy poodles, miniature schnauzers, Yorkshire terriers, dachshunds. Another predisposed dog breed is the Cavalier King Charles Spaniel. He is a special breed in that he often suffers from mitral endocardiosis at a young age. Large dogs are by far less frequently affected.

Signs of disease at an early stage:

Cardiac murmur: This cardiac murmur is audible to the veterinarian with the help of the stethoscope, even before the owner notices any changes in his own pet. This is why this disease can possibly be detected during routine examinations, such as vaccination examinations. Signs of disease in the further course: Coughing, increased breathing frequency, shortness of breath, listlessness, poor performance, lack of appetite, short phases of loss of consciousness: Causes: due to very irregular heartbeat, or severe coughing or as a result of a tear in the left atrium. According to the known technology, three diagnostics solutions are known:
X-ray
   Heart size: At first there is an enlargement of the heart shadow in the area of the left atrium and later also in the area of the left ventricle.
   Displacement of the left primary bronchus.
   Another important task of the X-ray image is the assessment of the pulmonary vessels and the lung field. If the pulmonary veins are congested, this is an indication for therapy. If a pulmonary edema is present, an alveolar opacity, usually in the hilus region, can be shown.
   Pulmonary congestion: At first the pulmonary veins appear congested, later pulmonary edema (water on the lungs) can be diagnosed.
ECG
   The ECG mainly diagnoses cardiac arrhythmias. It is an important diagnostic criterion, as dogs with mitral valve disease can get arrhythmias. Whether an ECG is useful is ultimately decided by the cardiologist, but one should always be taken if an arrhythmia or additional heart sounds are detected during monitoring.
Heart ultrasound
   The size of the atrium and ventricle can be measured so that any magnification can be reliably determined.
   The ability of the heart muscle to contract can be measured.
   In addition, colour Doppler echocardiography can be used to quantify the extent of the insufficiency.

According to embodiments of the present invention it is possible to automatically differentiate between pathological and healthy cardiac murmurs of animals or non-human mammals, in particular dogs. The sounds were auscultated per dog at four different positions (front left, back left, front right, back right). From the sound recordings, various characteristics are calculated both in the time and frequency domain, which serve as input for several machine learning algorithms after dividing the total data set into training and test data set. The classification between pathological and heart-healthy sounds unfortunately did not promise satisfactory results. For this reason, the classification was initially limited to 2 classes. These consist of recordings from dogs with healthy hearts and from dogs with mitral valve insufficiency (MR). MR is a heart valve defect that leads to blood flowing back from the left ventricle into the left atrium. With the algorithm Decision Trees, the classification of dogs weighing less than 20 kg achieved an accuracy of 84%, a precision of 81% and a recall of 81% (first test results). It is expected that the accuracy will increase. By use of additional sound samples an increase to 93% has been achieved. It should be noted that the data set is very small. There are breeds which are only represented by recordings from dogs with MR. The use of the feature “breed” would lead to falsified results and has therefore not been used. Thus, embodiments enable, for example, diagnosis of heart diseases in animals or non-human mammals, in particular dogs, having a simple setup (e.g. digital stethoscope and smartphone), inexpensive, quick to perform (low stress for the animal). This, further, enables beneficially telemedical examination.

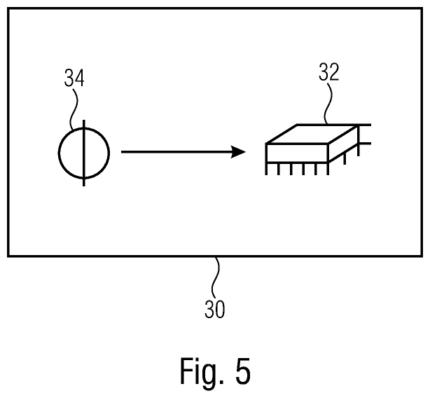

By use of the above-discussed approach, a simple apparatus can be formed. The apparatus is illustrated by FIG. 5. FIG. 5 shows the apparatus 30 comprising at least a processor 32 and a microphone 34. The microphone signal can be digitalized using an ADC. In a simple configuration, the apparatus can be formed without a microphone, and can then instead have an interface for receiving the audio signal. This configuration is not shown. The processor 32 is configured to determine the audio pattern repeated within the acoustic signal and to determine a window length for a plurality of windows. The window lengths divide a time period of an acoustic signal into the plurality of windows (evenly or unevenly). The processer is further configured to window the acoustic signal (starting from the window lengths) and to obtain the plurality of windows. Furthermore, the processor can be configured to perform the analysis, e.g., by feature extraction of the pattern within the plurality of windows. The entire apparatus can be implemented as a smart device/smartphone the analysis can be performed in an even manner. A more sophisticated implementation can be an apparatus as a stethoscope or a digital stethoscope.

According to embodiments, the above discussed apparatus can be implemented by a smart device, like a smart phone, tablet PC or other device comprising a processor 32. By use of the processor 32 the method or at least some method steps as defined above or defined in context of below embodiments can be executed. The method may, for example, be implemented as a software, application or algorithm for the smart device.

According to embodiments, a report, e. g. a report on the diagnosis may be output by the apparatus/stethoscope. For example, the report may comprise a diagnosis describing the determined disease/determined murmur disease. The report may be summed up to a kind of traffic light report having three-colors: yellow, red and green. Green may mean that no disease/murmur has been found so that the animal/non-human mammal/dog is in good condition. Yellow may mean that there is the danger/high probability of a murmur/disease. Yellow may additionally indicate that a further monitoring/further analysis of the animal/non-human mammal/dog is required. The red color may indicate that a murmur/disease has been found so that a treatment of the animal/non-human mammal/dog is required/suggested.

According to a different embodiment the report may be as follows:
Green light: It is very unlikely that the animal/non-human mammal/dog has a heart murmur. The heart sound is rather physiological.
Yellow Light: The heart sound differs slightly from a physiological sound. The animal/non-human mammal/dog may suffer from a heart disease and/or vascular disease other than a mitral valve leakage/disease. Please also consider repeating the auscultation.
Red light: It is very likely that the animal/non-human mammal/dog has a mitral valve murmur typical for a mitral valve heart disease. The murmur is staged into a loud heart murmur which is considered a clinically relevant heart murmur: it is recommended to conduct further examinations, such as a cardiac check up including either a chest x-ray or a echocardiography in order to determine whether the heart is enlarged and needs therapy (stage B2). Option B: the heart sound is staged into a soft heart murmur: a cardiac check-up every 6 months or annually is recommended.

According to embodiments, a simple summary can be output. An example for such a summery can be as follows:

“This is not a medical diagnosis. A vet visit is recommended to get a medical diagnosis. Heart murmur detection revealed the following:
with a probability of 95% this is a Mitral regurgitation (MMVD).
with a probability of 78% it is Pulmonic Stenosis (PS)
. . . ”

According to further embodiments, another kind of report is also possible. It should be noted that this report/diagnosis is generated automatically.

Below, further embodiments will be discussed in context of clauses.

Clause 1: A method (100) for analyzing (150) an acoustic signal (12a, 12b and 12c) having a time period (T0 to T6) and comprising a plurality of repeated audio patterns, comprising the following steps:
receiving (110) an audio signal (10, 10′, 10″, 10′, 10″ ″) comprising the acoustic signal (12a, 12b and 12c);
determining (120) the audio patterns repeated within the acoustic signal (12a, 12b and 12c);
determining (120) a window length for a plurality of windows (14a, 14b, 14c, 14a′ to 14o′, 14a″ to 14o″), wherein the window length divides the time period (T0 to T6) of the acoustic signal (12a, 12b and 12c) into the plurality of windows (14a, 14b, 14c, 14a′ to 14o′, 14a″ to 14o″); and
windowing (140) the acoustic signal to obtain the plurality of windows (14a, 14b, 14c, 14a′ to 14o′, 14a″ to 14o″).

Clause 2: The method (100) according to clause 1, wherein the method (100) comprises the further step of analyzing (150) the respective windows (14a, 14b, 14c, 14a′ to 14o′, 14a″ to 14o″).

Clause 3: The method (100) according to clause 2, wherein the further step of analyzing (150) comprises the step of performing a feature extraction to obtain one or more extracted features describing the respective pattern.

Clause 4: The method (100) according to clause 3, wherein the features to be extracted are out of the group comprising name feature, time domain feature and/or frequency domain feature; and/or wherein the feature to be extracted is out of the group comprising a maximum, a mean, median, standard deviation, variance, skewness, kurtosis, mean absolute deviation, quantile 25th, quantile 75th, entropy, zero crossing rate, crest factor, duration of a first peak and/or second peak within the pattern, duration between the first peak and the second peak within the pattern, duration between the second peak of a first pattern and the first peak of a subsequent pattern, mel frequency cepstral coefficients, pitch chroma, spectral flatness, spectral kurtosis, spectral skewness, spectral slope, spectral entropy, dominant frequency, bandwidth, spectral centroid, spectral flux, spectral roll off, class information, severity information, position information, race information, weight information, additional information and/or other parameters or a combination thereof; and/or wherein the step of feature extraction comprises the step of redefining the value range for the one or more extracted features so that the value range for the one or more extracted features is defined between a minimum value or 0 and a maximum value or 1.

Clause 5: The method (100) according to any one of the previous clauses, wherein the repeated audio patterns are equal to each other, substantially equal to each other, similar to each other, comprise one or more peaks of a comparable shape of the respective amplitude plotted over the time and/or comprise one or more peaks of a comparable shape of the amplitude plotted over the time and comparable amplitude values at the respective point of time within the window length.

Clause 6: The method (100) according to any one of the previous clauses, wherein the window length is equal.

Clause 7: The method (100) according to any one of the previous clauses, wherein the window length is determined based on the frequency of the repetition of the repeated pattern.

Clause 8: The method (100) according to any one of the previous clauses, wherein the method (100) further comprises the step of ignoring one or more windows (14a, 14b, 14c, 14a′ to 14o′, 14a″ to 14o″) without an audio pattern similar or equal to the plurality of repeated audio patterns.

Clause 9: The method (100) according to any one of the previous clauses, wherein each repeated audio pattern is defined by one or more peaks; and/or wherein each repeated audio pattern is defined by one or more peaks in combination with a basis level, wherein the one or more peaks have an amplitude value which is at least five times larger than the basis level; and/or wherein each repeated audio pattern is defined by a systole and/or diastole.

Clause 10: The method (100) according to any one of the previous clauses, wherein the method (100) comprises the step of normalizing the audio signal (10, 10′, 10″, 10′″, 10″ ″).

Clause 11: The method (100) according to any one of the previous clauses, wherein the step of determining (120) the audio patterns, determining (120) a window length and the windowing (140) are performed automatically and/or are performed by use artificial intelligence.

Clause 12: The method (100) according to clause 11, wherein the steps are performed by use of a decision tree algorithm, a random forest algorithm, a naive bayes algorithm, an adaboost algorithm, an algorithm implemented by a neuronal net and/or a support vector machine algorithm.

Clause 13: The method (100) according to any one of the previous clauses, wherein the audio signal (10, 10′, 10″, 10′″, 10″ ″) is a record of a heartbeat sequence of a dog and/or a record of a heart murmur sequence of a dog.

Clause 14: Apparatus (30) for analyzing (150) an acoustic signal (12a, 12b and 12c) having a time period (T0 to T6) and comprising a plurality of repeated audio patterns, the apparatus (30) comprises:
an interface for receiving (110) the audio signal (10, 10′, 10″, 10′″, 10″″) comprising the acoustic signal (12a, 12b and 12c); and
a processor (32) which is configured to determine the audio pattern repeated within the acoustic signal (12a, 12b and 12c) and to determine a window length for a plurality of windows (14a, 14b, 14c, 14a′ to 14o′, 14a″ to 14o″), wherein the window length divides the time period (T0 to T6) of the acoustic signal (12a, 12b and 12c) into the plurality of windows (14a, 14b, 14c, 14a′ to 14o′, 14a″ to 14o″); and to window the acoustic signal (12a, 12b and 12c) to obtain the plurality of windows (14a, 14b, 14c, 14a′ to 14o′, 14a″ to 14o″).

Clause 15: System for performing an analysis comprising the apparatus (30) according to clause 14 and a microphone (34) or advantageously the apparatus (30) according to clause 14 and a stethoscope comprising a microphone (34) or more advantageously the apparatus (30) according to clause 14 and a digital stethoscope comprising a microphone (34).

Clause 16: Computer program having a program code comprising instructions for performing the method (100) according to any one of the clauses 1 to 13.

Although some aspects have been described in the context of an apparatus, it is clear that these aspects also represent a description of the corresponding method, where a block or device corresponds to a method step or a feature of a method step. Analogously, aspects described in the context of a method step also represent a description of a corresponding block or item or feature of a corresponding apparatus. Some or all of the method steps may be executed by (or using) a hardware apparatus, like for example, a microprocessor, a programmable computer or an electronic circuit. In some embodiments, some one or more of the most important method steps may be executed by such an apparatus.

Depending on certain implementation requirements, embodiments of the invention can be implemented in hardware or in software. The implementation can be performed using a digital storage medium, for example a floppy disk, a DVD, a Blu-Ray, a CD, a ROM, a PROM, an EPROM, an EEPROM or a FLASH memory, having electronically readable control signals stored thereon, which cooperate (or are capable of cooperating) with a programmable computer system such that the respective method is performed. Therefore, the digital storage medium may be computer readable.

Some embodiments according to the invention comprise a data carrier having electronically readable control signals, which are capable of cooperating with a programmable computer system, such that one of the methods described herein is performed.

Generally, embodiments of the present invention can be implemented as a computer program product with a program code, the program code being operative for performing one of the methods when the computer program product runs on a computer. The program code may for example be stored on a machine readable carrier.

Other embodiments comprise the computer program for performing one of the methods described herein, stored on a machine readable carrier.

In other words, an embodiment of the inventive method is, therefore, a computer program having a program code for performing one of the methods described herein, when the computer program runs on a computer.

A further embodiment of the inventive methods is, therefore, a data carrier (or a digital storage medium, or a computer-readable medium) comprising, recorded thereon, the computer program for performing one of the methods described herein. The data carrier, the digital storage medium or the recorded medium are typically tangible and/or non-transitionary.

A further embodiment of the inventive method is, therefore, a data stream or a sequence of signals representing the computer program for performing one of the methods described herein. The data stream or the sequence of signals may for example be configured to be transferred via a data communication connection, for example via the Internet.

A further embodiment comprises a processing means, for example a computer, or a programmable logic device, configured to or adapted to perform one of the methods described herein.

A further embodiment comprises a computer having installed thereon the computer program for performing one of the methods described herein.

A further embodiment according to the invention comprises an apparatus or a system configured to transfer (for example, electronically or optically) a computer program for performing one of the methods described herein to a receiver. The receiver may, for example, be a computer, a mobile device, a memory device or the like. The apparatus or system may, for example, comprise a file server for transferring the computer program to the receiver.

In some embodiments, a programmable logic device (for example a field programmable gate array) may be used to perform some or all of the functionalities of the methods described herein. In some embodiments, a field programmable gate array may cooperate with a microprocessor in order to perform one of the methods described herein. Generally, the methods may be performed by any hardware apparatus.

While this invention has been described in terms of several embodiments, there are alterations, permutations, and equivalents which will be apparent to others skilled in the art and which fall within the scope of this invention. It should also be noted that there are many alternative ways of implementing the methods and compositions of the present invention. It is therefore intended that the following appended claims be interpreted as including all such alterations, permutations, and equivalents as fall within the true spirit and scope of the present invention.

1. A method for analyzing an acoustic signal comprising a time period and comprising a plurality of repeated audio patterns, comprising:
receiving an audio signal comprising the acoustic signal, wherein the audio signal is a record of a heartbeat sequence of an animal, advantageously a non-human mammal, more advantageously a dog, and/or a record of a heart murmur sequence of an animal, advantageously a non-human mammal, more advantageously a dog;
determining the audio patterns repeated within the acoustic signal;
determining a window length for a plurality of windows, wherein the window length divides the time period of the acoustic signal into the plurality of windows; wherein determining the window length is performed for each window of the plurality of windows separately; and
windowing the acoustic signal to acquire the plurality of windows;
wherein determining the audio patterns, determining a window length and the windowing are performed automatically.

2. The method according to claim 1, wherein the method further comprises analyzing the respective windows.

3. The method according to claim 2, wherein analyzing comprises performing a feature extraction to acquire one or more extracted features describing the respective pattern.

4. The method according to claim 3, wherein the features to be extracted are out of the group comprising name feature, time domain feature and/or frequency domain feature; and/or
wherein the feature to be extracted is out of the group comprising a maximum, a mean, median, standard deviation, variance, skewness, kurtosis, mean absolute deviation, quantile 25th, quantile 75th, entropy, zero crossing rate, crest factor, duration of a first peak and/or second peak within the pattern, duration between the first peak and the second peak within the pattern, duration between the second peak of a first pattern and the first peak of a subsequent pattern, mel frequency cepstral coefficients, pitch chroma, spectral flatness, spectral kurtosis, spectral skewness, spectral slope, spectral entropy, dominant frequency, bandwidth, spectral centroid, spectral flux, spectral roll off, class information, severity information, position information, race information, weight information, additional information and/or other parameters or a combination thereof; and/or
wherein the feature extraction comprises redefining the value range for the one or more extracted features so that the value range for the one or more extracted features is defined between a minimum value or 0 and a maximum value or 1.

5. The method according to claim 2, wherein the method comprises outputting a report on the analysis; or
wherein the method comprises outputting a report on the analysis, wherein the report comprises an information on a disease or a murmur of the animal, advantageously the non-human mammal, more advantageously the dog.

6. The method according to claim 1, wherein the repeated audio patterns are equal to each other, substantially equal to each other, similar to each other, comprise one or more peaks of a comparable shape of the respective amplitude plotted over the time and/or comprise one or more peaks of a comparable shape of the amplitude plotted over the time and comparable amplitude values at the respective point of time within the window length.

7. The method according to claim 1, wherein the window length is equal.

8. The method according to claim 1, wherein the window length is determined based on the frequency of the repetition of the repeated pattern.

9. The method according to claim 1, wherein the method further comprises ignoring one or more windows without an audio pattern similar or equal to the plurality of repeated audio patterns.

10. The method according to claim 1, wherein each repeated audio pattern is defined by one or more peaks; and/or
wherein each repeated audio pattern is defined by one or more peaks in combination with a basis level, wherein the one or more peaks comprise an amplitude value which is at least five times larger than the basis level; and/or
wherein each repeated audio pattern is defined by a systole and/or diastole.

11. The method according to claim 1, wherein the method comprises normalizing the audio signal.

12. The method according to claim 1, wherein determining the audio patterns, determining a window length and the windowing are performed by use artificial intelligence.

13. The method according to claim 12, wherein the steps are performed by use of a decision tree algorithm, a random forest algorithm, a naive bayes algorithm, an adaboost algorithm, and/or a support vector machine algorithm.

14. The method according to claim 1, wherein the acoustic signal comprises the heartbeat sequence of an animal, advantageously a non-human mammal, more advantageously a dog, the heartbeat sequences forming the plurality of repeated audio patterns.

15. The method according to claim 1, wherein determining the window length comprises determining a borderline between two repeated audio patterns so as to determine the window length for the respective window; and/or
wherein determining the window lengths comprises determining a characteristic feature, a pulse, peak, pattern, systole, and/or diastole of a window to determine a beginning of a window and to determine the respective feature, pulse, peak, pattern, systole and/or diastole of a subsequent window to determine the end of said window; and/or
wherein determining the window lengths comprises determining the window lengths by determining a beginning and an end of a window.

16. The method according to claim 1, wherein the window lengths is varied over time and/or wherein the window lengths is varied from a first window of the plurality of windows to a subsequent window of the plurality of windows.

17. The method according to claim 1, wherein the window lengths is varied dependent on a heartbeat rate of the animal, advantageously the non-human mammal, more advantageously the dog; and/or
wherein the method further comprises determining the heartbeat rate.

18. An apparatus for analyzing an acoustic signal comprising a time period and comprising a plurality of repeated audio patterns, the apparatus comprises:
an interface for receiving the audio signal comprising the acoustic signal; the audio signal is a record of a heartbeat sequence of an animal, advantageously a non-human mammal, more advantageously a dog and/or a record of a heart murmur sequence of an animal, advantageously a non-human mammal, more advantageously a dog; and
a processor which is configured to determine the audio pattern repeated within the acoustic signal and to determine a window length for a plurality of windows, wherein the window length divides the time period of the acoustic signal into the plurality of windows, wherein the processor determines the window length for each window of the plurality of windows separately; and to window the acoustic signal to acquire the plurality of windows;
wherein determining the audio patterns, determining a window length and the windowing are performed automatically.

19. A system for performing an analysis comprising the apparatus according to claim 18 and a microphone or advantageously the apparatus according to claim 18 and a stethoscope comprising a microphone or more advantageously the apparatus according to claim 18 and a digital stethoscope comprising a microphone.

20. A non-transitory digital storage medium having stored thereon a computer program for performing a method for analyzing an acoustic signal comprising a time period and comprising a plurality of repeated audio patterns, comprising:
receiving an audio signal comprising the acoustic signal, wherein the audio signal is a record of a heartbeat sequence of an animal, advantageously a non-human mammal, more advantageously a dog, and/or a record of a heart murmur sequence of an animal, advantageously a non-human mammal, more advantageously a dog;
determining the audio patterns repeated within the acoustic signal;
determining a window length for a plurality of windows, wherein the window length divides the time period of the acoustic signal into the plurality of windows; wherein determining the window length is performed for each window of the plurality of windows separately; and
windowing the acoustic signal to acquire the plurality of windows;
wherein determining the audio patterns, determining a window length and the windowing are performed automatically,
when said computer program is run by a computer.
